(19) United States
(12) Patent Application Publication
(10) Pub. No.: US 2024/0099796 A1
(43) Pub. Date: Mar. 28, 2024

(54) FLEXIBLE TUBE, DRIVING MECHANISM, CONTROL SYSTEM DRIVING THE SAME AND CONTROL METHOD CONTROLLING THE SAME

(71) Applicant: INDUSTRIAL TECHNOLOGY RESEARCH INSTITUTE, Hsinchu (TW)

(72) Inventors: Hao-Yan WU, Tainan City (TW); Chin-Chi HSIAO, Taoyuan City (TW); Chien-Yu WU, Yunlin County (TW); Shu HUANG, Hsinchu County (TW)

(73) Assignee: INDUSTRIAL TECHNOLOGY RESEARCH INSTITUTE, Hsinchu (TW)

(21) Appl. No.: 17/993,820

(22) Filed: Nov. 23, 2022

(30) Foreign Application Priority Data
Sep. 22, 2022 (TW) 111135908

(51) Int. Cl. A61B 34/00; A61B 1/00; A61B 1/005; A61B 1/008; A61B 34/30

(52) U.S. Cl. CPC A61B 34/71; A61B 1/00133; A61B 1/0055; A61B 1/0057; A61B 1/008; A61B 34/30; A61B 2034/301

(57) ABSTRACT

A flexible tube includes a first connecting portion and a second connecting section. The second connecting section and the first connecting section are integrally connected to each other. The first connecting section has a first end surface, and the second connecting section has a second end surface, wherein there is an acute angle between the first end surface and the second end surface.

This application claims the benefit of Taiwan application Serial No. 111135908, filed Sep. 22, 2022, the subject matter of which is incorporated herein by reference.

### TECHNICAL FIELD

The invention relates to a flexible tube, a driving mechanism, a control system driving the same and a control method controlling the same.

### BACKGROUND

A conventional flexible tube includes a plurality of connecting sections. Generally speaking, after the connecting sections are individually manufactured, they are connected together by means of snapping, pivoting, etc. However, such connecting approach makes the overall manufacturing process of the flexible tube too complicated. Therefore, it is one goal of those skilled in the art to propose a new flexible tube to improve the aforementioned problems.

### SUMMARY

According to an embodiment, a flexible tube is provided. The flexible tube includes a first connecting section and a second connecting section. The second connecting section is integrally connected with the first connecting section. The first connecting section has a first end surface, and the second connecting section has a second end surface, and there is an acute angle between the first end surface and the second end surface based on the first connecting section and the second connecting section being in a straight state.

According to another embodiment, a driving mechanism is provided. The driving mechanism is adapted for driving a flexible tube. The flexible tube includes a first connecting section and a second connecting section integrally connected with the first connecting section, the first connecting section has a first end surface, the second connecting section has a second end surface, there is an acute angle between the first end surface and the second end surface based on the first connecting section and the second connecting section being in a straight state, the flexible tube is connected to a connecting tube, a driving wire passes through the flexible tube and the connecting tube, and the driving mechanism includes a first driving module and a second driving module. The first driving module is connected to the driving wire and configured for driving the driving wire to control the flexible tube to bend. The second driving module connected to the connecting tube and configured for driving the connecting tube to rotate for driving the flexible tube to rotate.

According to another embodiment, a control system is provided. The control system includes a flexible tube, a driving mechanism and a controller. The flexible tube includes a first connecting section and a second connecting section. The second connecting section is integrally connected with the first connecting section. The first connecting section has a first end surface, and the second connecting section has a second end surface, and there is an acute angle between the first end surface and the second end surface based on the first connecting section and the second connecting section being in a straight state. The driving mechanism is configured for connecting the flexible tube. The controller electrically is connected to the driving mechanism and configured for controlling the driving mechanism to drive the flexible tube.

According to another embodiment, a control method is provided. The control method is adapted for driving a flexible tube. The flexible tube comprises a first connecting section, a second connecting section and a driving wire, the second connecting section is integrally connected with the first connecting section, the first connecting section has a first end surface, the second connecting section has a second end surface, there is an acute angle between the first end surface and the second end surface based on the first connecting section and the second connecting section being in a straight state, the driving wire passes through the first connecting section and the second connecting section and interferes with the first connecting section and the second connecting section, and the control method includes the following steps: pushing the drive wire toward a direction close to the flexible tube to increase a bending degree of the flexible tube; and pulling back the drive wire toward a direction away from the flexible tube to reduce the bending degree of the flexible tube.

The above and other aspects of the disclosure will become better understood with regard to the following detailed description of the preferred but non-limiting embodiment (s). The following description is made with reference to the accompanying drawings.

### BRIEF DESCRIPTION OF THE DRAWINGS

The above objects and advantages of the invention will become more readily apparent to those ordinarily skilled in the art after reviewing the following detailed description and accompanying drawings, in which:

FIGS. 1 and 2 show schematic diagrams of the flexible tube viewed from different viewing angles according to an embodiment of the present disclosure;

FIGS. 3 and 4 show schematic views of elevation views of the flexible tube of FIG. 1 viewed from different viewing angles;

FIG. 5 shows a schematic diagram of a cross-sectional view of the flexible tube of FIG. 4 along a direction 5-5′;

FIG. 6 shows a schematic diagram of a cross-sectional view of the flexible tube of FIG. 4 along a direction 6-6′;

FIG. 7 shows a schematic diagram of a control system according to an embodiment of the present disclosure;

FIG. 8A shows a schematic diagram of a control system according to an embodiment of the present disclosure;

FIG. 8B shows a schematic diagram of a driving mechanism of FIG. 8A; and

FIG. 9 shows a flowchart of a control method according to an embodiment of the present disclosure.

In the following detailed description, for purposes of explanation, numerous specific details are set forth in order to provide a thorough understanding of the disclosed embodiments. It will be apparent, however, that one or more embodiments could be practiced without these specific details. In other instances, well-known structures and devices are schematically shown in order to simplify the drawing.

### DETAILED DESCRIPTION OF PREFERRED EMBODIMENTS

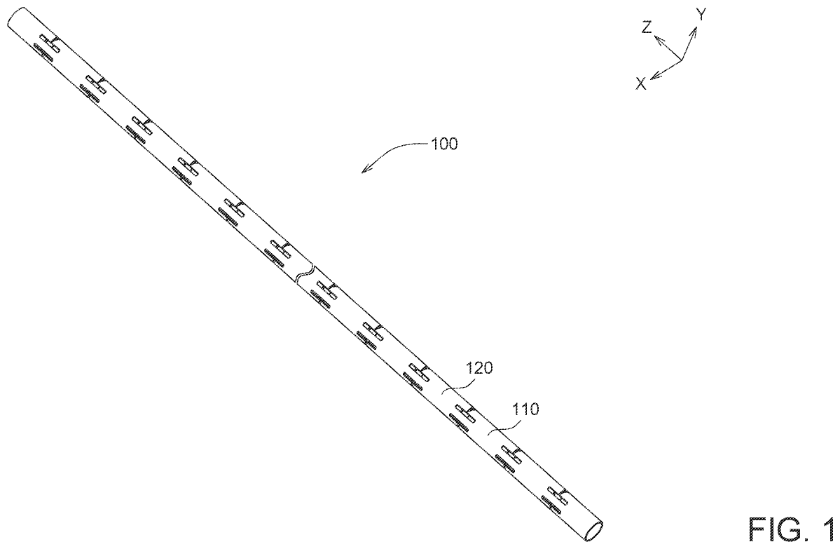

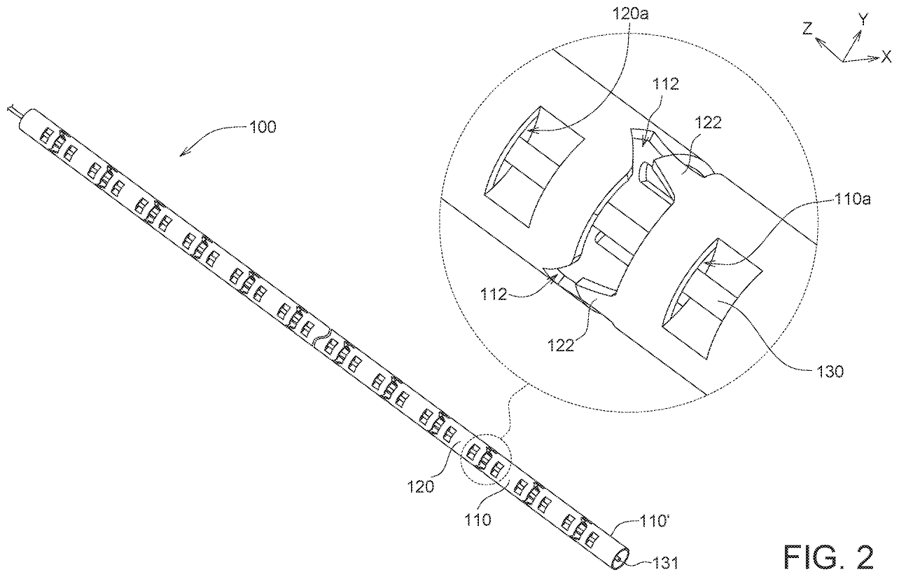

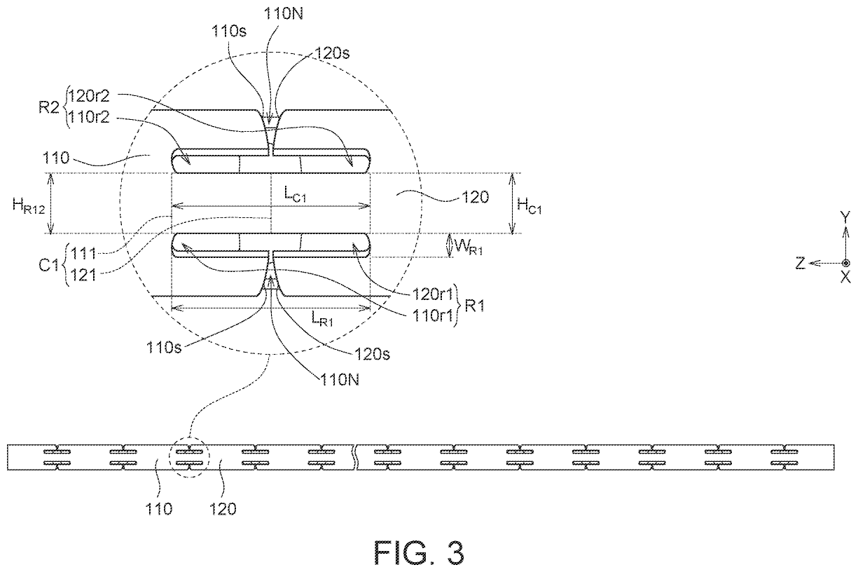

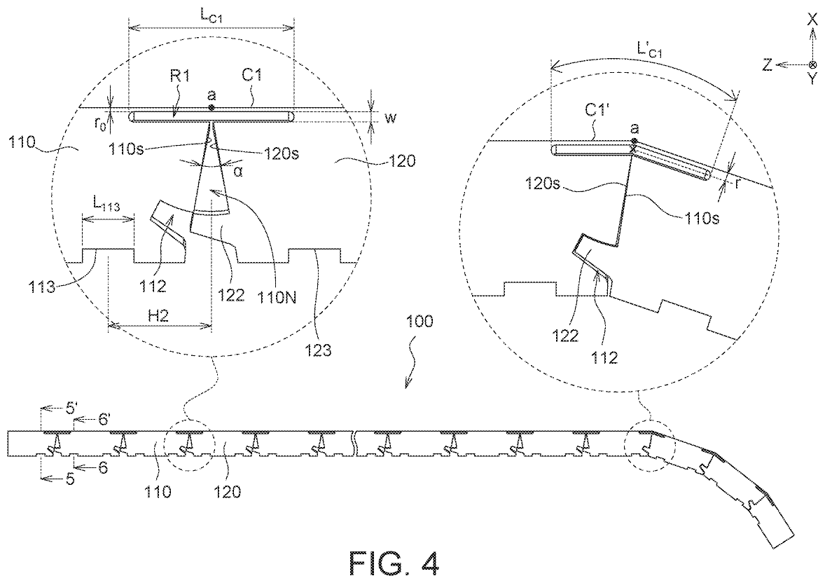

Referring to FIGS. 1 to 6, FIGS. 1 and 2 show schematic diagrams of the flexible tube 100 viewed from different viewing angles according to an embodiment of the present disclosure, FIGS. 3 and 4 show schematic views of elevation views of the flexible tube 100 of FIG. 1 viewed from different viewing angles, FIG. 5 shows a schematic diagram of a cross-sectional view of the flexible tube 100 of FIG. 4 along a direction 5-5′, and FIG. 6 shows a schematic diagram of a cross-sectional view of the flexible tube 100 of FIG. 4 along a direction 6-6′. To avoid the excessive complexity of the drawings, a driving wire 130 is not shown in FIGS. 1, 3 and 4.

The flexible tube 100 has flexibility and may extend in a curved to-be-detected object. In an embodiment, the flexible tube 100 may be used with an endoscope (not shown) to observe a structure within the to-be-detected object, such as tissues, defects or lesions in the to-be-detected object. The flexible tube 100 may be applied to medical treatment, and the aforementioned to-be-detected object is, for example, a tissue in a human body. The flexible tube 100 may also be applied in industry, and the aforementioned to-be-detected object is, for example, an industrial product, such as a pipe line; however, such exemplification is not meant to be for limiting.

The flexible tube 100 includes a plurality of connecting sections, adjacent two of the connecting sections are integrally connected (for example, formed from a single, contiguous piece of a material), and one of the connecting sections has a first end surface, and an adjacent-one of the connecting sections has a second end surface. When the first connecting section and the second connecting section are in a straight state (straight shape), there is an acute angle between the first end surface and the second end surface. As a result, the connecting sections are connected to each other, and the adjacent two connecting sections may move relative to each other within a range of the acute angle, so that the flexible tube 100 has (or provides) a bending freedom.

The following is an example of the adjacent two connecting sections (for example, the first connecting section 110 and the second connecting section 120) of the flexible tube 100, and other adjacent two connecting sections (which may also be regarded as the first connecting section 110 and the second connecting section 120 respectively) have the same or similar features, and the similarities will not be repeated again.

As shown in FIGS. 1 to 4, the second connecting section 120 and the first connecting section 110 are integrally connected. As shown in FIG. 3, the first connecting section 110 has a first end surface 110s, and the second connecting section 120 has a second end surface 120s. As shown in FIG. 4, when the first connecting section 110 and the second connecting section 120 are in a straight state, there is an acute angle α between the first end surface 110s and the second end surface 120s. As a result, the first connecting section 110 and the second connecting section 120 may relatively move within the range of the acute angle α, so that the flexible tube 100 has a bending degree of freedom, for example, the bending degree of freedom around the −X axis in FIG. 4. The X-axis, Y-axis and Z-axis shown in the figure are perpendicular to each other, wherein the Z-axis is, for example, parallel to a long axis of the flexible tube 100 when it is in straight state, and the Y-axis is, for example, parallel to a normal direction of a center point of a connecting area C1.

As shown in FIG. 4, through the slope design of the first end surface 110s and/or the slope design of the second end surface 120s, the acute angle α ranges, for example, between 5 degrees and 40 degrees, greater or smaller when the first connecting section 110 and the second connecting section 120 are in a straight state. When the first connecting section 110 and the second connecting section 120 relatively rotate until the first end surface 110s and the second end surface 120s are in contact, the acute angle α is 0 degree, for example. In addition, as shown in FIG. 3, there is a notch 110N between the first end surface 110s and the second end surface 120s, and the acute angle α is an angle of the gap 110N.

As shown in FIGS. 1 and 3, due to the second connecting section 120 and the first connecting section 110 being integrally connected without being joined by additional means such as snapping or pivoting, the overall fabrication of the flexible tube 100 may be simplified. Furthermore, the second connecting section 120 and the first connecting section 110 is not formed separately, but the second connecting section 120 and the first connecting section 110 are an integral structure before the second connecting section 120 and the first connecting section 110 are formed, and the second connecting section 120 and the first connecting section 110 are not completely separated from each other after the second connecting section 120 and the first connecting section 110 are formed. As a result, there is no obvious connection interface between the second connecting section 120 and the first connecting section 110. The flexible tube 100 is, for example, a tube formed by an integral molding process. The aforementioned “integral molding process” is, for example, a laser engraving technique. For example, a complete (without hollow pattern) tube may be provided first, and then the structure of the first connecting section 110 and the structure of the second connecting section 120 of the flexible tube 100 as shown in FIG. 1 may be engraved on the tube by laser, wherein the first connecting section 110 and the second connecting section 120 are kept connected (for example, not disconnected), or the first connecting section 110 and the second connecting section 120 are only kept connected in the connecting area C1, the rest are disconnected. Laser engraving may process a base tube with a small diameter, for example, a minimum diameter of 0.5 millimeters (mm), but may also be greater than 0.5 mm. If the flexible tube 100 is operated in the pulmonary bronchus, it may be designed to have a smaller diameter. If the flexible tube 100 is applied to a colonoscope, it may be designed to have a larger diameter, for example, 12 mm. In terms of material, it may be selected for medical purposes. For example, the aforementioned base tube (flexible tube 100) may be formed of a material including, for example, metal such as nickel, titanium or a combination thereof, or stainless steel, wherein nickel-titanium memory alloy has springback property. If it not limited to medical use, other metal material may also be used. In terms of shape, the base tube may be, for example, a cylindrical tube, but may also be an elliptical tube or a polygonal tube.

As shown in FIG. 3, the first connecting section 110 and the second connecting section 120 have a first cutting slot 110r1 and a second cutting slot 120r1. The first cutting slot 110r1 is connected to the second cutting slot 120r1. In the present embodiment, the first cutting slot 110r1 and the second cutting slot 120r1 are connected to form a single cutting slot R1 having the straight shape. In another embodiment, the single cutting slot R1 may have a curved shape, or a combined shape of a straight line and a curved line. The first connecting section 110 and the second connecting section 120 include a first connecting portion 111 and a second connecting portion 121 respectively. The first connecting portion 111 and the second connecting portion 121 are connected to each other and adjacent to the first cutting slot 110r1 and the second cutting slot 120r1.

As shown in FIG. 3, the first connecting section 110 has a third cutting slot 110r2, and the second connecting section 120 has a fourth cutting slot 120r2. The third cutting slot 110r2 and the fourth cutting slot 120r2 are connected to each other. In the present embodiment, the third cutting slot 110r2 and the fourth cutting slot 120r2 are connected to form a single cutting slot R2 having the straight shape. In another embodiment, the single cutting slot R2 may have a curved shape, or a combined shape of a straight line and a curved line. The first connecting portion 111 and the second connecting portion 121 are connected to each other and are adjacent to the third cutting slot 110r2 and the fourth cutting slot 120r2.

As shown in FIG. 3, the first connecting portion 111 is formed between the first cutting slot 110r1 and the third cutting slot 110r2, and the second connecting portion 121 is formed between the second cutting slot 120r1 and the fourth cutting slot 120r2. In addition, the first cutting slot 110r1 and the third cutting slot 110r2 are arranged along the Y axis, the second cutting slot 120r1 and the fourth cutting slot 120r2 are also arranged along the Y axis, and the first connecting portion 111 and the second connecting portion 121 are connected along the Z axis. A distance HR12 between the single cutting slot R1 and the single cutting slot R2 is, for example, any real number ranging between 0.3 mm and 0.6 mm, such as 0.48 mm; however, such exemplification is not meant to be for limiting.

As shown in FIG. 3, due to the connecting portions (the first connecting portion 111 and/or the second connecting portion 121) are formed after the cutting slots (the first cutting slot 110r1, the second cutting slot 120r1, the third cutting slot 110r2 and/or The fourth cutting slot 120r2) is formed on the connecting portion and the cutting slot have a corresponding relationship in size. For example, a length LR1 of the single cutting slot R1 in a direction (for example, the Z-axis) is equal to an initial length L_{C1} of the connecting regions C1 of the first connecting portion 111 and the second connecting portion 121 in the same direction (for example, the Z-axis). The single cutting slot R2 has similar or the same features, and details are not described herein again. A distance HR12 between the single cutting slot R1 and the single cutting slot R2 in a direction (for example, the Y axis) is equal to a length H_{C1} of the connecting region C1 in the same direction (for example, the Y axis).

As shown in FIG. 3, the size, position and/or the number of the cutting slots (the first cutting slot 110rl, the second cutting slot 120r1, the third cutting slot 110r2 and/or the fourth cutting slot 120r2) may change the rigidity of the first connecting section 110 and the second connecting section 120. For example, the single cutting slot R1 further has a width W_{R1}, wherein the width W_{R1} and the length LR1 may determine the rigidity of the first connecting section 110 and the second connecting section 120. For example, the longer the length LR1 is, the smaller the rigidity (the greater the flexibility), and vice versa. The wider the width W_{R1} is, the smaller the rigidity is, and vice versa.

As shown in FIG. 3, the first end surface 110s may be connected to the first cutting slot 110r1, and the second end surface 120s may be connected to the second cutting slot 120r1 (that is, the notch 110N extends to the cutting slot). Similarly, the first end surface 110s may be connected to the third cutting slot 110r2, and the second end surface 120s may be connected to the fourth cutting slot 120r2 (that is, the notch 110N extends to the cutting slot). Due to the cutting slot communicating with the end surface (or the cutting slot communicating with the notch 110N), the rigidity of the first connecting section 110 and the second connecting section 120 may be reduced (compared to that the end surface and the cutting slot are not connected).

As shown in FIG. 4, the first connecting section 110 includes at least one first limiting portion (position-limited portion) 112, and the second connecting section 120 includes at least one second limiting portion 122. The first limiting portion 112 is recessed relative to the first end surface 110s, and the second limiting portion 122 protrudes relative to the second end surface 120s. In the present embodiment, the first limiting portion 112 is, for example, a mortise, and the second limiting portion 122 is, for example, a tenon. In another embodiment, the first limiting portion 112 may protrude relative to the first end surface 110s to form a tenon, and the second limiting portion 122 may be recessed relative to the second end surface 120s to form a mortise.

In an embodiment, the first limiting portion 112 is an integrally formed structure of the first connecting section 110 and/or the second limiting portion 122 is an integrally molded structure of the second connecting section 120. For example, the first limiting portion 112 and the second limiting portion 122 are formed by laser-engraving the base tube.

As shown in FIG. 4, the first limiting portion 112 and the second limiting portion 122 may be selectively separated or engaged. For example, when the first connecting section 110 and the second connecting section 120 are in a straight state, the first limiting portion 112 and the second limiting portion 122 are completely or partially separated. When the first connecting section 110 and the second connecting section 120 are in a bent state, the first limiting portion 112 and the second limiting portion 122 are completely engaged or partially engaged. By the first limiting portion 112 and the second limiting portion 122 being restrained to each other, it may prevent the first connecting section 110 and the second connecting section 120 from being displaced in the Y-axis during bending and/or may prevent the first connecting section 110 and the second connecting section 120 from being displaced in the X-axis during bending. As a result, the stability and/or positioning accuracy of the flexible tube 100 may be increased during bending.

As shown in FIGS. 5 and 6, the first connecting section 110 includes at least one third limiting portion 113. The third limiting portion 113 is connected to a first inner sidewall 110w of the first connecting section 110 and a first through hole 110a is formed between the third limiting portion 113 and the first inner sidewall 110w. The first through hole 110a extends, for example, along an axial direction of the first connecting section 110, for example, the Z-axis. Similarly, the second connecting section 120 includes at least one fourth limiting portion 123. The fourth limiting portion 123 is connected to a second inner sidewall 120w of the second connecting section 120 and a second through hole 120a is formed between the fourth limiting portion 123 and the second inner sidewall 120w. The second through hole 120a extends, for example, along an axial direction of the second connecting section 120, for example, the Z-axis. As shown in FIG. 4, a length L₁₁₃ of the third limiting portion 113 in a direction (for example, the Z-axis) ranges between, for example, 0.25 mm and 0.75 mm, and a distance H2 between the third limiting portion 113 and a center line of the notch 110N along a direction (for example, the Z-axis) ranges between, for example, 1.0 mm and 2.0 mm. As a result, it may prevent the first connecting section 110 and the second connecting section 120 from being completely separated from each other and may ensure that the flexible tube 100 has enough strength. The fourth limiting portion 123 has structural features similar to or the same as that of the third limiting portion 113, and details are not repeated again.

In an embodiment, the third limiting portion 113 is an integrally formed structure of the first connecting section 110 and/or the fourth limiting portion 123 is an integrally molded structure of the second connecting section 120. For example, the third limiting portion 113 and the fourth limiting portion 123 may be formed on the above-mentioned base tube by laser engraving.

As shown in FIGS. 2, 5 and 6, the flexible tube 100 further includes a driving wire 130. The driving wire 130 may pass through the first connecting section 110 and the second connecting section 120. For example, the driving wire 130 may pass through the first through hole 110a of the first connecting section 110 and the second through hole 120a of the second connecting section 120. The driving wire 130 interferes with the first connecting section 110 and/or the second connecting section 120. For example, the first through hole 110a has a first inner diameter D11 which is smaller than an outer diameter d of the driving wire 130. As a result, the driving wire 130 interferes with the first through hole 110a, and the driving wire 130 may drive the first connecting section 110 and the second connecting section 120 to relatively bend (for example, rotate about the X-axis) through an interference resistance between the through hole and the driving wire 130 when the driving wire 130 slides relative to the flexible tube 100. The second through hole 120a has a second inner diameter D21 which is smaller than the outer diameter d of the driving wire 130. As a result, the driving wire 130 interferes with the second through hole 120a, and the driving wire 130 may drive the first connecting section 110 and the second connecting section 120 to relatively bend (for example, rotate about the X-axis) through an interference resistance between the through hole and the driving wire 130 when the driving wire 130 slides relative to the flexible tube 100. Compared with the driving wire 130 interfering with the through hole, the interference resistance between the driving wire 130 and the flexible tube 100 is greater due to the driving wire 130 interfering with both of the first through hole 110a and the second through hole 120a. In addition, in the present embodiment, although the number of the third limiting portion 113 is single and the number of the fourth limiting portion 123 is single, the technical efficacy (described later) of multiple bending DOF (degrees of freedom) (for example, the bending DOF around the X-axis and the bending DOF around the Y-axis) may be archived by using the driving method of the present disclosed embodiment.

As shown in FIG. 2, after the driving wire 130 passes through all the connecting sections, it may be fixed to the last connecting section. For example, the driving wire 130 has one end 131 which may be fixed to an inner side wall of the last connecting section 110′ by using, for example, bonding, welding, snapping, locking, etc. In an embodiment, the drive line 130 may be formed of, for example, metal. In an embodiment, the driving wire 130 is a multi-strand wire strand, for example, which is formed of 19 wire strands, each of which has a maximum outer diameter of 0.2 mm.

As shown in FIGS. 5 and 6, in an embodiment, the first inner diameter D11 is, for example, the largest inner diameter of the first through hole 110a along the Y-axis, and the second inner diameter D21 is, for example, the largest inner diameter of the second through hole 120a along the Y axis. The driving wire 130 is, for example, a wire having a circular cross section. In terms of size, the outer diameter d ranges between, for example, 0.2 mm and 0.5 mm, and the first inner diameter D11 and/or the second inner diameter D21 ranges between, for example, 0.2 mm and 0.5 mm.

As shown in FIGS. 5 and 6, the first through hole 110a further has a third inner diameter D12, and the second through hole 120a further has a fourth inner diameter D22. The third inner diameter D12 is, for example, the largest inner diameter of the first through hole 110a along the Z-axis, and the fourth inner diameter D22 is, for example, the largest inner diameter of the second through hole 120a along the Z-axis. The third inner diameter D12 is greater than the first inner diameter D11, and the fourth inner diameter D22 is greater than the second inner diameter D21. In terms of size, the third inner diameter D12 and/or the fourth inner diameter D22 ranges between, for example, 0.7 mm and 1.75 mm, and may be greater or smaller.

As shown in FIG. 4, the deformation of the flexible tube 100 may occur in or only in the connecting region C1. For example, the first connecting section 110 and the second connecting section 120 rotate around a rotation center “a”. The rotation center “a” is, for example, an intersection of an extension of the first end surface 110s and an extension of the second end surface 120s.

As shown in FIG. 4, referring to the following equations (1) to (4) at the same time, the equation (1) is deduced based on the principle that the volume of the connecting region C1 of the first connecting section 110 and the second connecting section 120 remains unchanged before and after deformation, wherein L_{C1} represents an initial length of the connecting region C1 in an initial state (the first connecting section 110 and the second connecting section 120 are in a straight state (unbent state)), and L′_{C1} represents a deformation length of the connecting region C1′ after bending. When the first connecting section 110 and the second connecting section 120 relatively rotate, the connecting area C1 is elongated, and thus the deformation length L′_{C1} is greater than the initial length L_{C1}. Equation (2) is a relational expression between the deformation length L′_{C1} of FIG. 4 and the initial length L_{C1}, wherein r represents a rotation radius of the first connecting section 110 and the second connecting section 120, and an unit of the acute angle α is degree. The Poisson's ratio p is, for example, 0.33 and the magnitude of strain γ is, for example, equal to 5% (or other real number less than 6%). Equations (1) and (2) may be generalized as equation (3).

$$\frac{{L}_{C 1}^{′ } ( p \cdot {L}_{C 1}^{′} + ( 1 - p ) \cdot {L}_{C 1} )}{{L}_{C 1}^{2}} < 1 + γ \qquad ( 1 )$$

$${L}_{C 1}^{′} = {L}_{C 1} + 2 π \cdot r \cdot \frac{α}{360} \qquad ( 2 )$$

$${L}_{C 1} > ( \sqrt{183.49} + 13.3 ) \frac{2 π r}{360} \times α \approx 0.47 \cdot r \cdot α \qquad ( 3 )$$

As shown in the following equations (4) and (5), in equation (4), S represents a length (full length) of the flexible tube 100, N represents the number of the connecting sections (the number of segments) of the flexible tube 100, and (α×N) represents the maximum bending angle of the flexible tube 100 (the bending angle of the flexible tube 100 when any adjacent two connecting sections relatively rotate and the first end surface 110s and the second end surface 120s of any adjacent two connecting sections are in contact (the acute angle α is equal to 0)). Equation (4) may ensure that the cutting slots of adjacent two connecting sections are not connected (if the cutting slots of adjacent two connecting sections are connected to each other, the flexible tube 100 is likely to fail). In equation (5), r₀ represents the minimum rotation radius of the flexible tube 100 which is a distance from the connecting area C1 of FIG. 4 to an edge of the single cutting slot R1, and w represents a width of the single cutting slot R1 of FIG. 4.

$${L}_{C 1} < ( S \cdot \frac{α}{α \times N} ) \qquad ( 4 )$$

$${r}_{0} \leq r \leq ( {r}_{0} + w ) \qquad ( 5 )$$

The embodiments of the present disclosure do not limit the values of the initial length L_{C1} and the rotational radius r of the connecting region C1, as long as the values satisfy the above equations (3) to (5). In an embodiment, assuming that r₀ is equal to 0.04 mm and the width w is equal to 0.1 mm, according to the above equation (5), the corresponding design value of the rotation radius r may be a real number ranging between 0.04 mm and 0.14 mm. In addition, in case of the length S of the flexible tube 100 being 50 mm, the number of the connecting sections being twelve (12 segments) and the acute angle α being 20 degrees (the corresponding maximum bending angle of the flexible tube is α×N1=240 degrees), according to the above equation (4), the relationship of L₀≤4 mm may be obtained, that is, the design value of the initial length L₀ is not greater than 4 mm, for example, 1.5 mm.

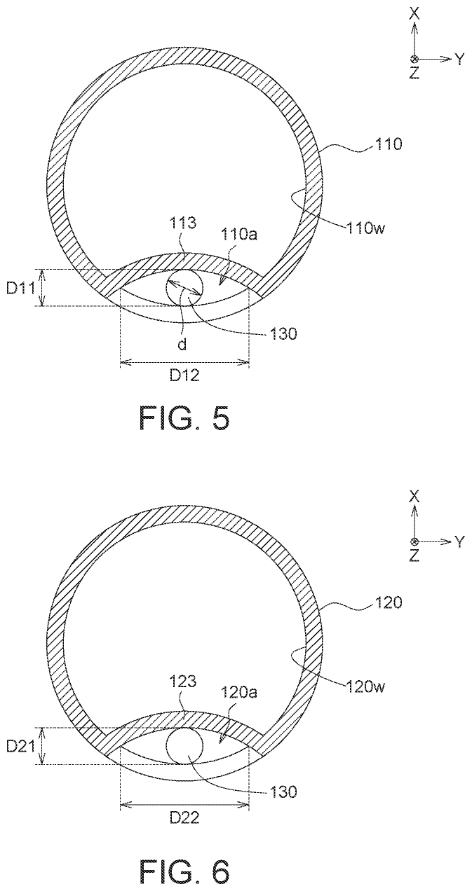

Referring to FIG. 7, FIG. 7 shows a schematic diagram of a control system 10 according to an embodiment of the present disclosure. The control system 10 includes a controller 11, a driving mechanism 200 and a flexible tube 100. The driving mechanism 200 is configured to connect the flexible tube 100. The controller 11 is electrically connected to the driving mechanism 200 for controlling the driving mechanism 200 to drive the flexible tube 100.

As shown in FIG. 7, the driving mechanism 200 is adapted to drive the flexible tube 100. The flexible tube 100 is connected to a connecting tube 140 and the driving wire 130 passes through the flexible tube 100 and the connecting tube 140. The driving mechanism 200 includes a first driving module 210 and a second driving module 220. The first driving module 210 is connected to the driving wire 130 and is configured for driving the driving wire 130 to drive the flexible tube 100 to bend. The second driving module 220 is connected to the connecting tube 140 and is configured for driving the connecting tube 140 to rotate and accordingly driving the flexible tube 100 to rotate. Due to the rotation and bending of the flexible tube 100, multiple bending DOF may be realized. Furthermore, the flexible tube 100 of the present disclosed embodiment may realize the technical effect of “single-wire (one driving wire 130) control to generate multiple DOF”. In addition, the flexible tube 100 is indirectly connected to the driving mechanism 200 through the connecting tube 140. As a result, the flexible tube 100 and the driving mechanism 200 are not directly connected, and accordingly it may prevent the flexible tube 100 from being damaged (for example, resulted from clamping or pinching) by the driving mechanism 200.

For example, as shown in FIG. 7, the flexible tube 100 may be driven to bend (for example, bend along −X axis) when the driving wire 130 is pulled back (for example, the driving wire 130 is pulled back along −Z axis). After the driving wire 130 is released (for example, the driving wire 130 is pushed out along +Z axis), the flexible tube 100 may be driven to return to a straight state (for example, the flexible tube 100 bends along +X axis). In another embodiment, the rotatable flexible tube 100 may be rotated around Z-axis by an angle, and then the driving wire 130 may be pulled back. At this time, the flexible tube 100 may bend around an axial-direction which is located on XY plane and there is an acute angle or an obtuse angle between X-axis and the axial-direction.

As shown in FIG. 7, the first driving module 210 includes a first driver 211 and a driving wheel 212. The driving wheel 212 is connected to the first driver 211. The drive wire 130 is wound around the drive wheel 212. Furthermore, the first driver 211 may drive the driving wheel 212 to rotate in a first rotation direction to pull back the driving wire 130, thereby driving the flexible tube 100 to bend or increasing the bending degree of the flexible tube 100, and the first driver 211 may drive the driving wheel 212 to rotate in a second rotation direction to release the driving wire 130, thereby driving the flexible tube 100 to return to a straight state or reducing the bending degree of the flexible tube 100. The aforementioned first rotation direction is, for example, one of clockwise and counterclockwise, and the second rotation direction is, for example, the other of clockwise and counterclockwise. The first driver 211 is, for example, a motor. The driving wheel 212 has a recess 212r, and the recess 212r may accommodate the driving wire 130 which is pulled back.

As shown in FIG. 7, the second driving module 220 includes a second driver 221 and a gear set 222. The gear set 222 is connected to the second driver 221. The connecting tube 140 is fixed to the gear set 222. The gear set 222 includes a first gear 2221 and a second gear 2222, wherein the second driver 221 is fixed to the first gear 2221, and the second gear 2222 meshes with the first gear 2221. The second driver 221 may drive the first gear 2221 to rotate, so as to drive the second gear 2222 to rotate, thereby driving the flexible tube 100 to self-rotate. The aforementioned connecting tube 140 may be connected to a center of the second gear 2222. For example, the center of the second gear 2222 has a connecting hole 2222a, and the connecting tube 140 may be disposed (for example, be engaged with) through the connecting hole 2222a.

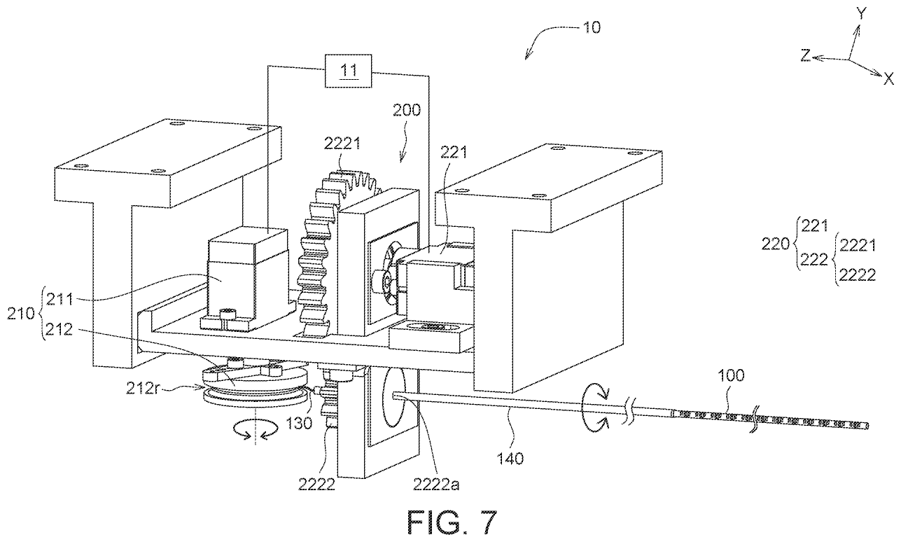

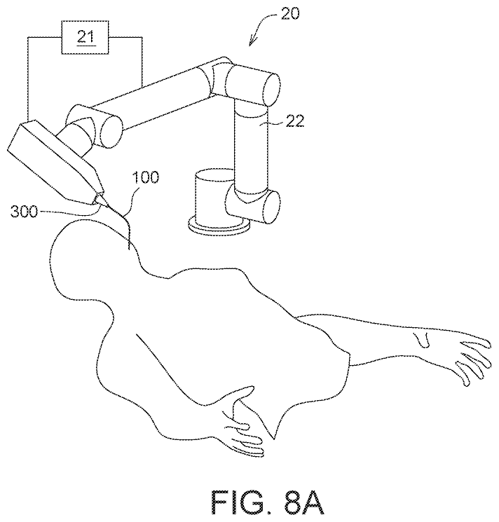

Referring to FIGS. 8A and 8B. FIG. 8A shows a schematic diagram of a control system 20 according to an embodiment of the present disclosure, and FIG. 8B shows a schematic diagram of a driving mechanism 300 of FIG. 8A. The control system 20 includes a controller 21, a driving mechanism 300, a robotic arm 22 and the flexible tube 100. The driving mechanism 300 may be disposed on the robotic arm 22. The robotic arm 22 may improve the DOF of manipulation, and it is helpful for human surgical operations. The driving mechanism 300 is configured to connect the flexible tube 100. The controller 21 is electrically connected to the driving mechanism 300 for controlling the driving mechanism 300 to drive the flexible tube 100. The driving mechanism 300 includes a first clamping element 310 and a second clamping element 320. The first clamping element 310 is configured for clamping the flexible tube 100 and rotating the flexible tube 100. For example, the first clamping element 310 may rotate the flexible tube 100 around Z-axis. The second clamping element 320 is configured for clamping the driving wire 130 and controlling the driving wire 130 to move relative to the flexible tube 100 (for example, moving towards the +/−Z axis) for controlling the flexible tube 100 to bend.

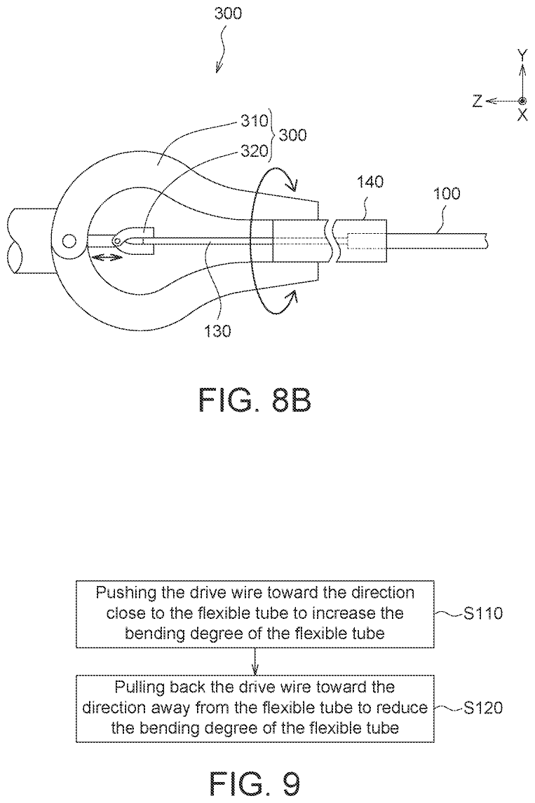

Referring to FIG. 9, FIG. 9 shows a flowchart of a control method according to an embodiment of the present disclosure. The control method may be performed by the aforementioned control system 10 or 20, or by the aforementioned driving mechanism 200 or 300. The following describes an example of the control method which is performed by the driving mechanism 200, for example.

In step S110, the driving mechanism 200 pushes the driving wire 130 toward the direction close to the flexible tube 100 for driving the flexible tube 100 to increase the bending degree.

In step S120, the driving mechanism 200 pulls back the driving wire 130 toward a direction away from the flexible tube 100 for driving the flexible tube 100 to reduce the bending degree.

In addition, the control method further includes the step of rotating (for example, around Z-axis) the flexible tube 100. Before rotating the flexible tube 100, the bending degree of the flexible tube 100 may be reduced first until the flexible tube 100 returns to a straight shape, and then the flexible tube 100 is rotated. Next, steps S110 and/or S120 may be repeated. In another embodiment, the flexible tube 100 may also be rotated under the flexible tube 100 being in a bent state. As described above, the control method of the embodiment of the present disclosure may realize the technical effect of “single-line (one driving wire 130) control to generate multiple bending DOF”.

To sum up, the embodiments of the present disclosure provide a flexible tube, a driving mechanism and a control system for driving the flexible tube, and a control method for controlling the flexible tube. The flexible tube includes a first connecting section and a second connecting section. The second connecting section is integrally connected with the first connecting section. The first connecting section has a first end surface, the second connecting section has a second end surface, and there is an acute angle formed between the first end surface and the second end surface. As a result, the connecting sections are connected to each other, and the adjacent two connecting sections may move relative to each other within the range of the acute angle, so that the flexible tube has a bending DOF. In another embodiment, the flexible tube further includes a driving wire, the driving wire passes through a plurality of the connecting sections of the flexible tube, and the bending degree of the flexible tube may be increased or reduced by pulling back or pushing out the driving wire. In an embodiment, the flexible tube may be directly or indirectly connected to a driving mechanism, and the driving mechanism may drive the flexible tube to bend and rotate (for example, self-rotate), so as to realize the technical effect of “single-line (one driving wire) control and generate multiple bending DOF”. In addition, as long as the driving mechanism may drive the flexible tube to rotate and may pull back or push out the driving wire which passes through the flexible tube, the embodiment of the present disclosure does not limit the specific structure of the driving mechanism.

It will be apparent to those skilled in the art that various modifications and variations could be made to the disclosed embodiments. It is intended that the specification and examples be considered as exemplary only, with a true scope of the disclosure being indicated by the following claims and their equivalents.

What is claimed is:

1. A flexible tube, comprising:
a first connecting section; and
a second connecting section integrally connected with the first connecting section;
wherein the first connecting section has a first end surface, and the second connecting section has a second end surface, and there is an acute angle between the first end surface and the second end surface based on the first connecting section and the second connecting section being in a straight state.

2. The flexible tube as claimed in claim 1, wherein the first connecting section and the second connecting section respectively have a first cutting slot and a second cutting slot, the first cutting slot and the second cutting slot are connected to each other, the first connecting section and the second connecting section respectively comprises a first connecting portion and a second connecting portion, the first connecting portion and the second connecting portion are connected to each other and adjacent to the first cutting slot and the second cutting slot.

3. The flexible tube as claimed in claim 2, wherein the first connecting section further has a third cutting slot, the second connecting section further has a fourth cutting slot, the first connecting portion of the first connecting section is formed between the first cutting slot and the third cutting slot, and the second connecting portion of the second connecting section is formed between the second cutting slot and the fourth cutting slot.

4. The flexible tube as claimed in claim 1, wherein the first connecting section comprises a first limiting portion, the second connecting section comprises a second limiting portion, the first limiting portion is protruded or recessed relative to the first end surface, the second limiting portion is recessed or protruded relative to the second end surface, and the first limiting portion and the second limiting portion are selectively separated or engaged.

5. The flexible tube as claimed in claim 1, wherein the first connecting section and the second connecting section respectively comprises a third limiting portion and a fourth limiting portion, the third limiting portion is connected to a first inner sidewall of the first connecting section, there is a first through hole formed between the third limiting portion and the first inner sidewall, the fourth limiting portion is connected to a second inner sidewall of the second connecting section, there is a second through hole formed between the fourth limiting portion and the second inner sidewall.

6. The flexible tube as claimed in claim 5, further comprising:
a driving wire passing through the first through hole and the second through hole;
wherein the first through hole has a first inner diameter, the first inner diameter is smaller than an outer diameter of the driving wire, the second through hole has a second inner diameter, and the second inner diameter is smaller than the outer diameter of the driving wire.

7. A driving mechanism, adapted for driving a flexible tube, wherein the flexible tube comprises a first connecting section and a second connecting section integrally connected with the first connecting section, the first connecting section has a first end surface, the second connecting section has a second end surface, there is an acute angle between the first end surface and the second end surface based on the first connecting section and the second connecting section being in a straight state, the flexible tube is connected to a connecting tube, a driving wire passes through the flexible tube and the connecting tube, and the driving mechanism comprises:
a first driving module connected to the driving wire and configured for driving the driving wire to control the flexible tube to bend; and
a second driving module connected to the connecting tube and configured for driving the connecting tube to rotate for driving the flexible tube to rotate.

8. The driving mechanism as claimed in claim 7, wherein the first driving module comprises:
a first driver; and
a driving wheel connected to the first driver;
wherein the driving wire is wound around the driving wheel.

9. The driving mechanism as claimed in claim 7, wherein the second driving module comprises:
a second driver; and
a gear set connected to the second driver;
wherein the connecting tube is fixed to the gear set.

10. A control system, comprising:
a flexible tube comprising:
a first connecting section; and
a second connecting section integrally connected with the first connecting section;
wherein the first connecting section has a first end surface, and the second connecting section has a second end surface, and there is an acute angle between the first end surface and the second end surface based on the first connecting section and the second connecting section being in a straight state;
a driving mechanism configured for connecting the flexible tube; and
a controller electrically connected to the driving mechanism and configured for controlling the driving mechanism to drive the flexible tube.

11. The control system as claimed in claim 10, further comprising:
a drive wire passing through the flexible tube;
wherein the driving mechanism comprises:
a first clamping element configured for clamping the flexible tube and rotating the flexible tube; and
a second clamping element configured for clamping the driving wire and controlling the driving wire to move relative to the flexible tube.

12. The control system as claimed in claim 10, further comprising a driving wire and a connecting tube, wherein the driving wire passes through the flexible tube and the connecting tube, the flexible tube is connected to the connecting tube, and the driving mechanism comprises:
a first driving module connected to the driving wire and configured for driving the driving wire to control the flexible tube to bend; and
a second driving module connected to the connecting tube and configured for driving the connecting tube to rotate, so as to drive the flexible tube to rotate.

13. The control system as claimed in claim 10, wherein the first connecting section and the second connecting section respectively have a first cutting slot and a second cutting slot, the first cutting slot and the second cutting slot are connected to each other; the first connecting section and the second connecting section respectively comprises a first connecting portion and a second connecting portion, the first connecting portion and the second connecting portion are connected to each other and adjacent to the first cutting slot and the second cutting slot.

14. The control system as claimed in claim 13, wherein the first connecting section further has a third cutting slot, the second connecting section further has a fourth cutting slot, the first connecting portion of the first connecting section is formed between the first cutting slot and the third cutting slot, and the second connecting portion of the second connecting section is formed between the second cutting slot and the fourth cutting slot.

15. The control system as claimed in claim 10, wherein the first connecting section comprises a first limiting portion, the second connecting section comprises a second limiting portion, the first limiting portion is protruded or recessed relative to the first end surface, the second limiting portion is recessed or protruded relative to the second end surface, and the first limiting portion and the second limiting portion are selectively separated or engaged.

16. The control system as claimed in claim 10, wherein the first connecting section and the second connecting section respectively comprises a third limiting portion and a fourth limiting portion, the third limiting portion is connected to a first inner sidewall of the first connecting section, there is a first through hole formed between the third limiting portion and the first inner sidewall, the fourth limiting portion is connected to a second inner sidewall of the second connecting section, there is a second through hole formed between the fourth limiting portion and the second inner sidewall.

17. The control system as claimed in claim 16, wherein the flexible tube further comprises a driving wire, the driving wire passes through the first through hole and the second through hole, the first through hole has a first inner diameter, the first inner diameter is smaller than an outer diameter of the driving wire, the second through hole has a second inner diameter, and the second inner diameter is smaller than the outer diameter of the driving wire.

18. A control method, adapted for driving a flexible tube, wherein the flexible tube comprises a first connecting section, a second connecting section and a driving wire, the second connecting section is integrally connected with the first connecting section, the first connecting section has a first end surface, the second connecting section has a second end surface, there is an acute angle between the first end surface and the second end surface based on the first connecting section and the second connecting section being in a straight state, the driving wire passes through the first connecting section and the second connecting section and interferes with the first connecting section and the second connecting section, and the control method comprises:
pushing the drive wire toward a direction close to the flexible tube to increase a bending degree of the flexible tube; and
pulling back the drive wire toward a direction away from the flexible tube to reduce the bending degree of the flexible tube.

19. The control method as claimed in claim 18, further comprising:
rotating the flexible tube.

20. The control method as claimed in claim 18, further comprising:
pulling back the drive wire toward the direction away from the flexible tube until the flexible tube is in the straight state; and
rotating the flexible tube when the flexible tube is in the straight state.
